(19) United States
(12) Patent Application Publication
(10) Pub. No.: US 2005/0043345 A1
(43) Pub. Date: Feb. 24, 2005

(54) Pharmaceutical composition for the prevention and treatment of addiction in a mammal

(75) Inventors: Jotham Wadsworth Coe, Niantic, CT (US); Philip A. Iredale, Clinton, CT (US); Stanton Furst McHardy, Coventry, RI (US); Stafford McLean, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/870,821

(22) Filed: Jun. 17, 2004

Related U.S. Application Data
(60) Provisional application No. 60/497,372, filed on Aug. 22, 2003.

(51) Int. Cl. A61K 31/4745; A61K 31/407; A61K 31/18

(52) U.S. Cl. 514/300; 514/306; 514/602; 514/412

(57) ABSTRACT

Pharmaceutical compositions are disclosed for the treatment of alcohol or cocaine dependence or addiction, tobacco dependence or addiction, reduction of alcohol withdrawal symptoms or aiding in the cessation or lessening of alcohol use or substance abuse or other behavioral dependencies including gambling. The pharmaceutical compositions are comprised of a therapeutically effective combination of an opioid receptor antagonist and an alpha2delta ligand and a pharmaceutically acceptable carrier. The method of using these compounds is also disclosed.

### BACKGROUND OF THE INVENTION

The present invention relates to pharmaceutical compositions for the treatment of alcohol, cocaine, or tobacco dependence or addiction and behavior dependencies including gambling in a mammal (e.g. human) comprising an opioid receptor antagonist and an alpha2delta ligand. The compounds of the subject invention bind to opioid receptors (e.g. mu, kappa and delta opioid receptors). Compounds that bind to such receptors are likely to be useful in the treatment of diseases modulated by opioid receptors, for example irritable bowel syndrome; constipation; nausea; vomiting; and pruritic dermatoses, such as allergic dermatitis and atopy in animals and humans. Compounds that bind to opioid receptors have also been indicated in the treatment of eating disorders, opioid overdoses, depression, anxiety, schizophrenia, alcohol addiction, including alcohol abuse and dependency, sexual dysfunction, shock, stroke, spinal damage and head trauma. The present invention may be used to treat mammals (e.g. humans) for alcohol dependence or addiction and nicotine dependence or addiction; to palliate the effects of alcohol withdrawal, to enhance the outcomes of other alcohol cessation therapies and to treat substance abuse and behavioral dependencies e.g. gambling.

The opioid receptor antagonists bind to opioid receptor sites and can be used in combination with an alpha2delta ligand to treat addiction such as to alcohol, cocaine or tobacco, alcohol dependence, cocaine addiction or tobacco or alcohol dependence independently of other psychiatric illness or other behavioral dependencies, e.g. gambling.

Approximately 13.5 million individuals in the US suffer from alcohol abuse and dependence (AAD). Untreated alcoholics are among the highest users of US health care, consuming 15% of each health care dollar. In addition, the indirect costs associated with productivity loss, property damage, and premature death are estimated at $100 billion per year. Only 20% receive any treatment and less than 10% receive any drug treatment related to AAD. Yet it is increasingly viewed as a disease amenable to drug interventing.

### SUMMARY OF INVENTION

The present invention relates to a pharmaceutical composition for treating alcohol dependence or addiction, tobacco dependence or addiction, reducing alcohol withdrawal symptoms or aiding in the cessation or lessening of alcohol use or substance abuse or behavioral dependencies, including gambling, comprising:
(a) an opioid receptor antagonist or a pharmaceutically acceptable salt thereof;
(b) an alpha2delta ligand or pharmaceutically acceptable salt thereof; and
(c) a pharmaceutically acceptable carrier;
wherein the active agents “a” and “b” above are present in amounts that render the composition effective in treating alcohol dependence or addiction, tobacco dependence or addiction, reducing alcohol withdrawal symptoms or aiding in the cessation or lessening of alcohol use or substance abuse or behavioral dependencies.

The alpha2delta ligands are selected from:
3-Amino-5-methyl-octanoic acid;
3-Amino-5-methyl-nonanoic acid;
(3S,5R)-3-Amino-5-methyl-heptanoic acid;
(3S,5R)-3-Amino-5-methyl-octanoic acid;
(3S,5R)-3-Amino-5-methyl-nonanoic acid;
3-Amino-7-cyclopentyl-5-methyl-heptanoic acid;
3-Amino-7-cyclohexyl-5-methyl-heptanoic acid;
(3S,5R)-3-Amino-7-cyclopentyl-5-methyl-heptanoic acid;
(3S,5R)-3-Amino-7-cyclohexyl-5-methyl-heptanoic acid;
3-Amino-5-methyl-7-phenyl-heptanoic acid;
3-Amino-5-methyl-7-(2,4-difluoro-phenyl)-heptanoic acid;
3-Amino-8-(2,3-difluoro-phenyl)-5-methyl-octanoic acid;
3-Amino-8-(2,4-difluoro-phenyl)-5-methyl-octanoic acid;
2-Aminomethyl-4-methyl-heptanoic acid;
(2R,4R)-2-Aminomethyl-4-methyl-heptanoic acid;
(2R,4S)-2-Aminomethyl-4-methyl-heptanoic acid;
2-Aminomethyl-3-[1-4-methyl-pentyl)-cyclopropyl]-propionic acid;
2-Aminomethyl-4-ethyl-8-methyl-nonanoic acid;
2-Aminomethyl-3-(1-methyl-cyclopropy)-propionic acid;
2-Aminomethyl-4,4-dimethyl-8-methyl-nonanoic acid;
2-Aminomethyl-4-cyclohexyl-3-methyl-butyric acid;
2-Aminomethyl-4,6-dimethyl-heptanoic acid;
1-(aminomethyl)-cyclohexane acetic acid;
(1-aminomethyl-3-methylcyclohexyl) acetic acid;
(1-aminomethyl-3-methylcyclopentyl) acetic acid;
(1-aminomethyl-3,4-dimethylcyclopentyl)acetic acid.
(S)-3-(aminomethyl)-5-methyl hexanoic acid;
3-(1-aminoethyl)-5-methylheptanoic acid or 3-(1-aminoethyl)-5-methylhexanoic acid;
C-[1-(1H-Tetrazol-5-ylmethyl)-cycloheptyl]-methylamine;
(3S,4S)-(1-Aminomethyl-3,4-dimethyl-cyclopentyl)-acetic acid;
(3-amino-methyl-bicyclo[3.2.0]hept-3-yl)-acetic acid;
3-(1-aminomethyl-cyclohexylmethyl)-4H-[1,2,4]oxadiazol-5-one;
3-(1-aminomethyl-cycloheptylmethyl)-4H-[1,2,4]oxadiazol-5-one; and
3-(1-aminomethyl-cycloheptylmethyl)-4H-[1,2,4]oxadiazol-5-one hydrochloride.
tert-Butyl({2-[(4-bromophenyl)sulfanyl]ethyl}amino)acetate;
tert-Butyl({2-[(4-chlorophenyl)sulfanyl]ethyl}amino)acetate;
tert-Butyl {[2-(2,4-dichlorophenoxy)ethyl]amino}acetate;
tert-Butyl({2-[(4-chlorobenzyl)sulfanyl]ethyl}amino)acetate;
tert-Butyl {[2-(7-isoquinolinylsulfanyl)ethyl]amino}acetate;
({2-[(4-Chlorophenyl)sulfanyl]ethyl}amino)acetic acid;
({2-[(4-Bromophenyl)sulfanyl]ethyl}amino)acetic acid;
[(2-{[4-(Aminomethyl)phenyl]sulfanyl}ethyl)amino]acetic acid;
{[2-(2,4-Dichlorophenoxy)ethyl]amino}acetic acid;
({2-[(4-Chlorobenzyl)sulfanyl]ethyl}amino)acetic acid;
{[2-(7-Isoquinolinylsulfanyl)ethyl]amino}acetic acid;
Ethyl({2-[(4-chlorophenyl)sulfanyl]ethyl}amino)acetate;
[2-(4-chloro-phenoxy)-propylamino]-acetic acid tert-butyl ester;
[2-(4-chloro-phenoxy)-propylamino]-acetic acid hydrochloride salt;
[2-(4-Methylsufanyl-phenylsufanyl)-ethylamino]-acetic acid tert-butyl ester;
[2-(4-Methylsufanyl-phenylsufanyl)-ethylamino]-acetic acid hydrochloride salt;
(4-Phenyl-butylamino)-acetic acid methyl ester;
4-Phenylbutylamino acetic acid:hydrochloride salt;
[2-(3-Chloro-phenoxy)-butylamino]-acetic acid; dihydrochloride;
2-aminomethyl-5-chloro-benzoic acid;
2-aminomethyl-4,5-d ichloro-benzoic acid;
2-aminomethyl-3-bromo-benzoic acid;
2-aminomethyl-6-chloro-benzoic acid;
2-(1-aminoethyl)-benzoic acid;
2,3-dihydro-1H-isoindole-4-carboxylic acid;
3-(2-aminomethyl-5-chloro-phenyl)-4H-[1,2,4]oxadiazol-5-one;
(1R,5R,6S)-[6-(aminomethyl)bicyclo[3.2.0]hept-6-yl]acetic acid; and
(1α,3α,5α)-[3-(aminomethyl)bicyclo[3.2.0]hept-3-yl]acetic acid

In another more specific embodiment of this invention, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide; N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

The present invention also relates to a method of treating alcohol or cocaine dependence or addiction, tobacco dependence or addiction, reducing alcohol withdrawal symptoms or aiding in the cessation or lessening of alcohol use or substance abuse or behavioral dependencies including gambling, comprising:
(a) an opioid receptor antagonist or a pharmaceutically acceptable salt thereof; and
(b) an alpha2delta ligand or pharmaceutically acceptable salt thereof; and
(c) a pharmaceutically acceptable carrier;
wherein the active agents (a) and (b) above are present in amounts that render the composition effective in treating alcohol or cocaine dependence or addiction, tobacco dependence or addiction, reducing alcohol withdrawal symptoms or aiding in the cessation or lessening of alcohol use or substance abuse or behavioral dependencies.

The opioid receptor antagonist and the alpha2delta ligand are present in amounts that render the composition effective in the treatment of alcohol, cocaine or nicotine addiction, alcohol withdrawal symptoms, substance abuse or other behavioral dependencies including gambling.

In another more specific embodiment of this invention the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

Preferably, the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above.

The term “substance abuse”, as used herein, for example in “drug addiction” and “alcohol addiction”, unless otherwise indicated, refers to a maladaptive use of a substance, which may be either with physiological dependence or without. The term “substance abuse” thus includes both substance abuse (e.g. alcohol, nicotine, amphetamine, cocaine or an opioid, for example morphine, opium, or heroine, abuse) and substance dependence (e.g. alcohol, nicotine, amphetamine, cocaine or an opioid, for example morphine, opium, or heroine dependence). The maladaptive pattern of substance use may manifest itself in recurrent and significant adverse consequences related to the repeated use of the substance. The recurrent substance use may result in a failure to fulfill major role obligations at work, school, or home. The maladaptive use of a substance may involve continued use of the substance despite persistent or recurrent social or interpersonal problems caused or exacerbated by the effects of the substance (e.g., arguments with spouse, physical fights). The maladaptive pattern of substance use may involve clinically significant impairment or distress, for example manifested by tolerance for the substance, withdrawal symptoms, unsuccessful efforts to cut down or control the substance use, and/or taking larger amounts of the substance and/or taking amounts of the substance over a longer period than was intended. Substances to which an addiction may be formed include, but are not limited to, the drugs recited above (including alcohol), as well as others, for example benzodiazepines such as Valium®.

Behavioral dependencies as used here means enduring or persistent patterns of behavior which deviates markedly from the expectations of an individual's culture, is pervasive and inflexible, is stable over time, and leads to distress or impairment, and can include either Axis I or Axis II diagnoses (1994; DSM-IV, American Psychiatric Association). Such diagnoses may include, but are not limited to, substance abuse (nicotine, alcohol, narcotics, inhalants), gambling, eating disorders, and impulse control disorders.

The chemist of ordinary skill will recognize that certain compounds of this invention will contain one or more atoms, which may be in a particular stereochemical or geometric configuration, giving rise to stereoisomers and configurational isomers. All such isomers and mixture thereof are included in this invention. Hydrates of the compounds of this invention are also included.

The chemist of ordinary skill will recognize that certain combinations of heteroatom-containing substituent listed in this invention define compounds, which will be less stable under physiological conditions (e.g., those containing acetal or animal linkages). According, such compounds are less preferred.

### DETAILED DESCRIPTION OF THE INVENTION

In combination with the opioid receptor antagonist the invention includes a alpha2delta and a pharmaceutically acceptable salt thereof.

The particular opioid receptor ligands listed above, which can be employed in the methods and pharmaceutical compositions of this invention, can be made by processes known in the chemical arts, for example by the methods described in WO 03/035,622 published May 1, 2003 which is U.S. Ser. No. No. 10/278,142 and 60/462,651 filed Apr. 14, 2003 and 60/462,629 filed Apr. 14, 2003 and 60/462,605 filed Apr. 14, 2003 which are incorporated by reference in their entireties.

The invention also relates to alpha2deltal ligands. Several alpha2delta ligands are known. Gabapentin, a cyclic alpha2delta ligand, is now commercially available (Neurontin®, Warner-Lambert Company) and extensively used clinically for treatment of epilepsy and neuropathic pain. Such cyclic alpha2delta ligands are described in U.S. Pat. No. 4,024,175, which issued on May 17, 1977, and U.S. Pat. No. 4,087,544, which issued on May 2, 1978.

Other series of alpha2delta ligands are described in U.S. Pat. No. 5,563,175, which issued on Oct. 8, 1996, U.S. Pat. No. 6,316,638, which issued on Nov. 13, 2001, U.S. Provisional Patent Application 60/353,632, which was filed on Jan. 31, 2002, U.S. Provisional Patent Application 60/248,630, which was filed on Nov. 2, 2002, U.S. Provisional Patent Application 60/421,868, which was filed on Oct. 28, 2002, U.S. Provisional Patent Application 60/421,867, which was filed on Oct. 28, 2002, U.S. Provisional Patent Application 60/413,856, which was filed on Sep. 25, 2002, U.S. Provisional Patent Application 60/411,493, which was filed on Sep. 16, 2002, U.S. Provisional Patent Application 60/421,866, which was filed on Oct. 28, 2002, U.S. Provisional Patent Application 60/441,825, which was filed on Jan. 22, 2003, U.S. Provisional Patent Application 60/452,871, which was filed on Mar. 7, 2003, European Patent Application EP 1112253, which was published on Jul. 4, 2001, PCT Patent Application WO 99/08671, which was published on Feb. 25, 1999, and PCT Patent Application WO 99/61424, which was published on Dec. 2, 1999. These patents and applications are incorporated herein by reference in their entireties.

Some of the preparation methods useful for making the compounds of this invention may require protection of remote functionality (i.e., primary amine, secondary amine, carboxyl). The need for such protection will vary depending on the nature of the remote functionality and the conditions of the preparation methods. The need for such protection is readily determined by one skilled in the art, and is described in examples carefully described in the above cited applications. The starting materials and reagents for the opioid receptor ligands employed in this invention are also readily available or can be easily synthesized by those skilled in the art using conventional methods of organic synthesis. Some of the compounds used herein are related to, or are derived from compounds found in nature and accordingly many such compounds are commercially available or are reported in the literature or are easily prepared from other commonly available substances by methods which are reported in the literature.

Some of the opioid receptor ligands employed in this invention are ionizable at physiological conditions. Thus, for example some of the compounds of this invention are acidic and they form a salt with a pharmaceutically acceptable cation. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the opioid receptor ligands employed in this invention are basic, and they form a salt with a pharmaceutically acceptable anion. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, when the opioid receptor ligands employed in this invention form hydrates or solvates they are also within the scope of the invention.

Some of the compounds of this invention are chiral, and as such are subject to preparation via chiral synthetic routes, or separable by conventional resolution or chromatographic means. All optical forms of the compounds of this invention are within the scope of the invention.

The utility of the opioid receptor ligands employed in the present invention as medicinal agents in the treatment of nicotine dependence (such as tobacco dependence or addiction) in mammals (e.g. humans) is demonstrated by the activity of the compounds of this invention in conventional assays and, in particular the assays described below. These include neuronal nicotinic receptor binding, dopamine turnover. Such assays also provide a means whereby the activities of the compounds of this invention can be compared between themselves and with the activities of other known compounds. The results of these comparisons are useful for determining dosage levels in mammals, including humans, for the treatment of such diseases.

### Biological Assays

### Procedures

Biological Activity

Compounds of the subject invention have been found to display activity in opioid receptor binding assays selective for the mu, kappa and delta opioid receptors. Assays for mu, kappa and deltan opioid receptor binding can be performed according to the following procedure:

Affinity of a compound for the delta opioid receptor can be assessed using binding of the delta opioid receptor ligand [³H]-naltrindole to NG108-15 neuroblastoma-glioma cells according to modification of the protocol described in Law et al. (Law, P. Y., Koehler, J. E. and Loh, H. H., “Comparison of Opioid Inhibition of Adenylate Cyclase Activity in Neuroblastoma N18TG2 and Neuroblastoma X Glioma NG108-15 Hybrid Cell Lines”, Molecular Pharmacology, 21: 483-491 (1982)). Law et al. is incorporated herein in its entirety by reference. Affinity of a compound for the kappa opioid receptor can be assessed using binding of [³H]-bremazocine to kappa receptors as described in Robson, L. E., et al., “Opioid Binding Sites of the Kappa-type in Guinea-pig Cerebellum”, Neuroscience (Oxford), 12(2): 621-627 (1984). Robson et al. is incorporated herein it its entirety by reference. For assessment of a compound for mu opioid receptor activity, the mu receptor ligand [³H]-DAMGO (Perkin Elmer Life Sciences, Boston, Mass.; specific activity 55 Ci/mmol, 1.5 nM) is used with rat forebrain tissue. Briefly, the binding is initiated with the addition of a crude membrane preparation of rat forebrain tissue to 96-well polypropylene plates containing the radioligand [³H]-DAMGO and test compound, and are incubated for about 90 minutes at about 25° C. The assay is terminated by rapid filtration with 50 mM Tris HCl pH 7.4 onto Wallac Filtermat B and counted on a Betaplate reader (Wallac).

The data generated can be analyzed using IC₅₀ analysis software in Graphpad Prism. Ki values can be calculated using Graphpad Prism according to the following formula:
Ki=IC₅₀/1+[³H ligand]/K_{D}
where IC₅₀ is the concentration at which 50% of the ³H ligand is displaced by the test compound and K_{D} is the dissociation constant for the ³H ligand at the receptor site.

The Ki values of certain compounds of formula I of the Examples, as described, infra, in a mu opioid receptor binding assay to brain tissue such as that described above, were determined. All of the compounds tested in this manner were all found to have Ki values of about 800 nM or less for the mu opioid receptor.

The inhibition (%) of [³H]-DAMGO binding by certain compounds of formula I of the Examples, as described, infra, in a mu opioid receptor binding assay to brain tissue such as that described above, were determined. Most of the compounds tested at 100 nM were found to inhibit [³H]-DAMGO binding at the mu opioid receptor in a range of 10-100%.

### Pharmacological Testing of Alpha2delta

The biological activity of the alpha2delta ligands of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996;271:5776-5879). Result may be expressed in terms of μM or nM a26 binding affinity.

Biological Data

Compounds of the invention were tested in the radioligand binding assay descried within and were found to have binding affinities as follows:

| Example | α2δ | |
|---|---|---|
| 1 | 100 | nM |
| 5 | 270 | nM |
| 2 | 435 | nM |
| 4 | 383 | nM |
| 7 | 8 | μM |
| 9 | 1665 | nM |
| 8 | 987 | nM |
| 12 | 5406 | nM |
| 6 | 198 | nM |
| 10 | 507 | nM |
| 11 | 71 | nM |
| 20 | 59 | nM |

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising an opioid receptor antagonist as described above and a alpha2delta ligand as described above in a pharmaceutically acceptable carrier can be administered.

The amount and timing of compounds administered will, of course, be based on the judgement of the prescribing physician. Thus, because of patient to patient variability, the dosages given below are a guideline and the physician may titrate doses of the agent to achieve the activity that the physician considers appropriate for the individual patient. In considering the degree of activity desired, the physician must balance a variety of factors such as cognitive function, age of the patient, presence of preexisting disease, as well as presence of other diseases (e.g., cardiovascular). The following paragraphs provide preferred dosage ranges for the various components of this invention (based on average human weight of 70 kg).

In general, an effective dosage for the opioid receptor antagonist or a pharmaceutically acceptable salt thereof can be administered orally, transdermally (e.g., through the use of a patch), parenterally (e.g. intravenously), rectally, topically, or by inhalation. In general, the daily dosage for treating a disorder or condition as described herein using a compound of formula I will be about from about 0.01 to about 100 mg per kg, preferably from about 0.1 to about 10 mg per kg, of the body weight of the animal to be treated. As an example, a compound of the formula I, or a pharmaceutically acceptable salt thereof, can be administered for treatment to an adult human of average weight (about 70 kg) in a dose ranging from about 0.1 mg up to about 10 g per day, preferably from about 1 mg to about 1 g per day, in single or divided (i.e., multiple) portions. Variations based on the aforementioned dosage ranges may be made by a physician of ordinary skill taking into account known considerations such as the weight, age, and condition of the animal being treated, the severity of the affliction, and the particular route of administration chosen.

In general, an effective dosage for the alpha2delta ligand when used in the combination compositions and methods of this invention, is in the range of 0.001 to 200 mg/kg/day, preferably 0.005 to 10.0 mg/kg/day.

The compositions of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral or transdermal dosage form.

For oral administration a pharmaceutical composition can take the form of solutions, suspensions, tablets, pills, capsules, powders, and the like. Tablets containing various excipient such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the dependence of the subject being treated.

1. A pharmaceutical composition for treating alcohol or cocaine dependence or addiction, tobacco dependence or addiction, reducing alcohol withdrawal symptoms or aiding in the cessation or lessening of alcohol use or substance abuse or behavioral dependencies including gambling, comprising:
(a) an alpha2delta ligand or a pharmaceutically acceptable salt thereof;
(b) an opioid receptor antagonist or pharmaceutically acceptable salt thereof; and
(c) a pharmaceutically acceptable carrier
wherein the active agents “a” and “b” above are present in amounts that render the composition effective in treating alcohol or cocaine dependence or addiction, tobacco dependence or addiction, reducing alcohol withdrawal symptoms or aiding in the cessation or lessening of alcohol use or substance abuse or behavioral dependencies.

2. The pharmaceutical composition according to claim 1, wherein said opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-yl methyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

3. The pharmaceutical composition according to claim 1, wherein the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-ylmethyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-yl methyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-ylmethyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

4. The pharmaceutical composition according to claim 1, wherein said alpha2delta ligand is selected from:
3-Amino-5-methyl-octanoic acid;
3-Amino-5-methyl-nonanoic acid;
(3S,5R)-3-Amino-5-methyl-heptanoic acid;
(3S,5R)-3-Amino-5-methyl-octanoic acid;
(3S,5R)-3-Amino-5-methyl-nonanoic acid;
3-Amino-7-cyclopentyl-5-methyl-heptanoic acid;
3-Amino-7-cyclohexyl-5-methyl-heptanoic acid;
(3S,5R)-3-Amino-7-cyclopentyl-5-methyl-heptanoic acid;
(3S,5R)-3-Amino-7-cyclohexyl-5-methyl-heptanoic acid;
3-Amino-5-methyl-7-phenyl-heptanoic acid;
3-Amino-5-methyl-7-(2,4-difluoro-phenyl)-heptanoic acid;
3-Amino-8-(2,3-difluoro-phenyl)-5-methyl-octanoic acid;
3-Amino-8-(2,4-difluoro-phenyl)-5-methyl-octanoic acid;
2-Aminomethyl-4-methyl-heptanoic acid;
(2R,4R)-2-Aminomethyl-4-methyl-heptanoic acid;
(2R,4S)-2-Aminomethyl-4-methyl-heptanoic acid;
2-Aminomethyl-3-[1-4-methyl-pentyl)-cyclopropyl]-propionic acid;
2-Aminomethyl-4-ethyl-8-methyl-nonanoic acid;
2-Aminomethyl-3-(1-methyl-cyclopropy)-propionic acid;
2-Aminomethyl-4,4-dimethyl-8-methyl-nonarioic acid;
2-Aminomethyl-4-cyclohexyl-3-methyl-butyric acid;
2-Aminomethyl-4,6-dimethyl-heptanoic acid;
1-(aminomethyl)-cyclohexane acetic acid;
(1-aminomethyl-3-methylcyclohexyl) acetic acid;
(1-aminomethyl-3-methylcyclopentyl) acetic acid;
(1-aminomethyl-3,4-dimethylcyclopentyl) acetic acid.
(S)-3-(aminomethyl)-5-methylhexanoic acid;
3-(1-aminoethyl)-5-methylheptanoic acid or 3-(1-aminoethyl)-5-methylhexanoic acid;
C-[1-(1H-Tetrazol-5-ylmethyl)-cycloheptyl]-methylamine;
(3S,4S)-(1-Aminomethyl-3,4-dimethyl-cyclopentyl)-acetic acid;
(3-amino-methyl-bicyclo[3.2.0]hept-3-yl)-acetic acid;
3-(1-aminomethyl-cyclohexylmethyl)-4H-[1,2,4]oxadiazol-5-one;
3-(1-aminomethyl-cycloheptylmethyl)-4H-[1,2,4]oxadiazol-5-one; and
3-(1-aminomethyl-cycloheptylmethyl)-4H-[1,2,4]oxadiazol-5-one hydrochloride.

5. The pharmaceutical composition according claim 1, wherein said alpha2delta ligand is selected from:
tert-Butyl({2-[(4-bromophenyl)sulfanyl]ethyl}amino)acetate;
tert-Butyl({2-[(4-chlorophenyl)sulfanyl]ethyl}amino)acetate;
tert-Butyl {[2-(2,4-dichlorophenoxy)ethyl]amino}acetate;
tert-Butyl({2-[(4-chlorobenzyl)sulfanyl]ethyl}amino)acetate;
tert-Butyl {[2-(7-isoquinolinylsulfanyl)ethyl]amino}acetate;
({2-[(4-Chlorophenyl)sulfanyl]ethyl}amino)acetic acid;
({2-[(4-Bromophenyl)sulfanyl]ethyl}amino)acetic acid;
[(2-{[4-(Aminomethyl)phenyl]sulfanyl}ethyl)amino]acetic acid;
{[2-(2,4-Dichlorophenoxy)ethyl]amino}acetic acid;
({2-[(4-Chlorobenzyl)sulfanyl]ethyl}amino)acetic acid;
{[2-(7-Isoquinolinylsulfanyl)ethyl]amino}acetic acid;
Ethyl({2-[(4-chlorophenyl)sulfanyl]ethyl}amino)acetate;
[2-(4-chloro-phenoxy)-propylamino]-acetic acid tert-butyl ester;
[2-(4-chloro-phenoxy)-propylamino]-acetic acid hydrochloride salt;
[2-(4-Methylsufanyl-phenylsufanyl)-ethylamino]-acetic acid tert-butyl ester;
[2-(4-Methylsufanyl-phenylsufanyl)-ethylamino]-acetic acid hydrochloride salt;
(4-Phenyl-butylamino)-acetic acid methyl ester;
4-Phenylbutylamino acetic acid hydrochloride salt;
[2-(3-Chloro-phenoxy)-butylamino]-acetic acid; dihydrochloride;
2-aminomethyl-5-chloro-benzoic acid;
2-aminomethyl-4,5-dichloro-benzoic acid;
2-aminomethyl-3-bromo-benzoic acid;
2-aminomethyl-6-chloro-benzoic acid;
2-(1-aminoethyl)-benzoic acid;
2,3-dihydro-1H-isoindole-4-carboxylic acid;
3-(2-aminomethyl-5-chloro-phenyl)-4H-[1,2,4]oxadiazol-5-one;
(1R,5R,6S)-[6-(aminomethyl)bicyclo[3.2.1]hept-6-yl]acetic acid; and
(1α,3α,5α)-[3-(aminomethyl)bicyclo[3.2.0]hept-3-yl]acetic acid.

6. A method of treating a mammal which presents with alcohol, cocaine, or nicotine addiction, alcohol withdrawal symptoms, substance abuse or behavioral dependencies including gambling, comprising administering to said mammal:
(a) an opioid receptor antagonist or a pharmaceutically acceptable salt thereof; and
(b) an alpha2delta ligand or a pharmaceutically acceptable salt thereof; and
(c) a pharmaceutically acceptable carrier;
wherein the opioid receptor antagonist and the alpha2delta ligand are present in amounts that render the composition effective in the treatment of alcohol, cocaine, or nicotine addiction, alcohol withdrawal symptoms, substance abuse or behavior dependencies.

7. The method according to claim 6, wherein said opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

8. The method according to claim 6, wherein the opioid receptor antagonist is selected from:
2-methoxy-ethanesulfonic acid {3-[6-ethyl-3-(2-hydroxy-1,2,3,4-tetrahydro-naphthalen-2-ylmethyl)-3-aza-bicyclo[3.1.0]hex-6-yl]-phenyl}-amide;
N-[3-(6-ethyl-3-indan-2-ylmethyl-3-aza-bicyclo[3.1.0]hex-6-yl)-phenyl]-methanesulfonamide;
2-methoxy-ethanesulfonic acid [3-(6-ethyl-3-indan-2-yl methyl-3-aza-bicyclo[3.1.0]hex-6-yl)-phenyl]-amide;
N-{3-[6-ethyl-3-(2-hydroxy-indan-2-ylmethyl)-3-aza-bicyclo[3.1.0]hex-6-yl]-phenyl}-methanesulfonamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
N-(3-{6-ethyl-3-[3-(1-hydroxy-cyclohexyl)-propyl]-3-aza-bicyclo[3.1.0]hex-6-yl}-phenyl)-methanesulfonamide;
3-{3-[3-(1-hydroxy-cyclohexyl)-propyl]-8-methoxy-3-aza-bicyclo[3.2.1]oct-8-yl}-benzamide;
2-methoxy-ethanesulfonic acid {3-[6-ethyl-3-(2-hydroxy-indan-2-ylmethyl)-3-aza-bicyclo[3.1.0]hex-6-yl]-phenyl}-amide;
3-[6-ethyl-3-(2-hydroxy-indan-2-ylmethyl)-3-aza-bicyclo[3.1.0]hex-6-yl]-benzamide;
N-{3-[6-ethyl-3-(2-hydroxy-1,2,3,4-tetrahydro-naphthalen-2-ylmethyl)-3-aza-bicyclo[3.1.0]hex-6-yl]-phenyl}-methanesulfonamide;
3-[6-ethyl-3-(2-hydroxy-1,2,3,4-tetrahydro-naphthalen-2-ylmethyl)-3-aza-bicyclo[3.1.0]hex-6-yl]-benzamide;
N-{3-[2-(2-hydroxy-indan-2-ylmethyl)-2-aza-bicyclo[3.3.1]non-5-yl]-phenyl}-methanesulfonamide;
2-methoxy-ethanesulfonic acid {3-[3-(2-hydroxy-indan-2-ylmethyl)-8-methoxy-3-aza-bicyclo[3.2.1]oct-8-yl]-phenyl}-amide;
3-{6-ethyl-3-[3-(1-hydroxy-cyclohexyl)-propyl]-3-aza-bicyclo[3.1.0]hex-6-yl}-benzamide;
3-[6-ethyl-3-(1-hydroxy-3-phenyl-cyclobutylmethyl)-3-aza-bicyclo[3.1.0]hex-6-yl]-benzamide;
2-methoxy-ethanesulfonic acid (3-{6-ethyl-3-[3-(1-hydroxy-cyclohexyl)-propyl]-3-aza-bicyclo[3.1.0]hex-6-yl}-phenyl)-amide;
3-{1-[3-(1-hydroxy-cyclohexyl)-propyl]-3,4-dimethyl-piperidin-4-yl}-benzamide;
3-(1-indan-2-ylmethyl-3,4-dimethyl-piperidin-4-yl)-benzamide;
N-{3-[3-(2-hydroxy-indan-2-ylmethyl)-8-methoxy-3-aza-bicyclo[3.2.1]oct-8-yl]-phenyl}-methanesulfonamide;
3-[1-(1-hydroxy-3-phenyl-cyclobutylmethyl)-3,4-dimethyl-piperidin-4-yl]-benzamide;
3-(6-hthyl-3-indan-2-ylmethyl-3-aza-bicyclo[3.1.0]hex-6-yl)-benzamide;
N-(3-{3-[3-(1-hydroxy-cyclohexyl)-propyl]-8-methoxy-3-aza-bicyclo[3.2.1]oct-8-yl}-phenyl)-methanesulfonamide;
3-[1-(2-hydroxy-indan-2-ylmethyl)-3,4-dimethyl-piperidin-4-yl]-benzamide;
N-(3-{2-[3-(1-hydroxy-cyclohexyl)-propyl]-2-aza-bicyclo[3.3.1]non-5-yl}-phenyl)-methanesulfonamide;
3-[3-(1-hydroxy-3-phenyl-cyclobutylmethyl)-8-methoxy-3-aza-bicyclo[3.2.1]oct-8-yl]-benzamide;
3-[3-(2-hydroxy-indan-2-yl methyl)-8-methoxy-3-aza-bicyclo[3.2.1]oct-8-yl]-benzamide;
3-[2-(2-hydroxy-indan-2-ylmethyl)-2-aza-bicyclo[3.3.1]non-5-yl]-benzamide;
3-{2-[3-(1-hydroxy-cyclohexyl)-propyl]-2-aza-bicyclo[3.3.1]non-5-yl}-benzamide;
3-[2-(1-hydroxy-3-phenyl-cyclobutylmethyl)-2-aza-bicyclo[3.3.1]non-5-yl]-benzamide;
3-[2-(2-hydroxy-1,2,3,4-tetrahydro-naphthalen-2-ylmethyl)-2-aza-bicyclo[3.3.1]non-5-yl]-benzamide;
2-methoxy-ethanesulfonic acid {3-[2-(2-hydroxy-indan-2-ylmethyl)-2-aza-bicyclo[3.3.1]non-5-yl]-phenyl}-amide;
2-methoxy-ethanesulfonic acid (3-{2-[3-(1-hydroxy-cyclohexyl)-propyl]-2-aza-bicyclo[3.3.1]non-5-yl}-phenyl)-amide;
2-methoxy-ethanesulfonic acid {3-[2-(1-hydroxy-3-phenyl-cyclobutylmethyl)-2-aza-bicyclo[3.3.1]non-5-yl]-phenyl}-amide;
2-methoxy-ethanesulfonic acid {3-[2-(2-hydroxy-1,2,3,4-tetrahydro-naphthalen-2-ylmethyl)-2-aza-bicyclo[3.3.1]non-5-yl]-phenyl}-amide;
N-{3-[2-(1-hydroxy-3-phenyl-cyclobutylmethyl)-2-aza-bicyclo[3.3.1]non-5-yl]-phenyl}-methanesulfonamide; and
N-{3-[2-(2-hydroxy-1,2,3,4-tetrahydro-naphthalen-2-ylmethyl)-2-aza-bicyclo[3.3.1]non-5-yl]-phenyl}-methanesulfonamide.

9. The method according to claim 6, wherein the alpha2delta ligand is selected from:
3-Amino-5-methyl-octanoic acid;
3-Amino-5-methyl-nonanoic acid;
(3S,5R)-3-Amino-5-methyl-heptanoic acid;
(3S,5R)-3-Amino-5-methyl-octanoic acid;
(3S,5R)-3-Amino-5-methyl-nonanoic acid;
3-Amino-7-cyclopentyl-5-methyl-heptanoic acid;
3-Amino-7-cyclohexyl-5-methyl-heptanoic acid;
(3S,5R)-3-Amino-7-cyclopentyl-5-methyl-heptanoic acid;
(3S,5R)-3-Amino-7-cyclohexyl-5-methyl-heptanoic acid;
3-Amino-5-methyl-7-phenyl-heptanoic acid;
3-Amino-5-methyl-7-(2,4-difluoro-phenyl)-heptanoic acid;
3-Amino-8-(2,3-difluoro-phenyl)-5-methyl-octanoic acid;
3-Amino-8-(2,4-difluoro-phenyl)-5-methyl-octanoic acid;
2-Aminomethyl-4-methyl-heptanoic acid;
(2R,4R)-2-Aminomethyl-4-methyl-heptanoic acid;
(2R,4S)-2-Aminomethyl-4-methyl-heptanoic acid;
2-Aminomethyl-3-[1-4-methyl-pentyl)-cyclopropyl]-propionic acid;
2-Aminomethyl-4-ethyl-8-methyl-nonanoic acid;
2-Aminomethyl-3-(1-methyl-cyclopropy)-propionic acid;
2-Aminomethyl-4,4-dimethyl-8-methyl-nonanoic acid;
2-Aminomethyl-4-cyclohexyl-3-methyl-butyric acid;
2-Aminomethyl-4,6-dimethyl-heptanoic acid;
1-(aminomethyl)-cyclohexane acetic acid;
(1-aminomethyl-3-methylcyclohexyl) acetic acid;
(1-aminomethyl-3-methylcyclopentyl) acetic acid;
(1-aminomethyl-3,4-dimethylcyclopentyl) acetic acid.
(S)-3-(aminomethyl)-5-methylhexanoic acid;
3-(11-aminoethyl)-5-methylheptanoic acid or 3-(1-aminoethyl)-5-methylhexanoic acid;
C-[1-(1H-Tetrazol-5-ylmethyl)-cycloheptyl]-methylamine;
(3S,4S)-(1-Aminomethyl-3,4-dimethyl-cyclopentyl)-acetic acid;
(3-amino-methyl-bicyclo[3.2.0]hept-3-yl)-acetic acid;
3-(1-aminomethyl-cyclohexylmethyl)-4H-[1,2,4]oxadiazol-5-one;
3-(1-aminomethyl-cycloheptylmethyl)-4H-[1,2,4]oxadiazol-5-one; and
3-(1-aminomethyl-cycloheptylmethyl)-4H-[1,2,4]oxadiazol-5-one hydrochloride.

10. The method according to claim 6, wherein the alpha2delta ligand is selected from:
tert-Butyl({2-[(4-bromophenyl)sulfanyl]ethyl}amino)acetate;
tert-Butyl({2-[(4-chlorophenyl)sulfanyl]ethyl}amino)acetate;
tert-Butyl {[2-(2,4-dichlorophenoxy)ethyl]amino}acetate;
tert-Butyl({2-[(4-chlorobenzyl)sulfanyl]ethyl}amino)acetate;
tert-Butyl {[2-(7-isoquinolinylsulfanyl)ethyl]amino}acetate;
({2-[(4-Chlorophenyl)sulfanyl]ethyl}amino)acetic acid;
({2-[(4-Bromophenyl)sulfanyl]ethyl}amino)acetic acid;
[(2-{[4-(Aminomethyl)phenyl]sulfanyl}ethyl)amino]acetic acid;
{[2-(2,4-Dichlorophenoxy)ethyl]amino}acetic acid;
({2-[(4-Chlorobenzyl)sulfanyl]ethyl}amino)acetic acid;
{[2-(7-Isoquinolinylsulfanyl)ethyl]amino}acetic acid;
Ethyl({2-[(4-chlorophenyl)sulfanyl]ethyl}amino)acetate;
[2-(4-chloro-phenoxy)-propylamino]-acetic acid tert-butyl ester;
[2-(4-chloro-phenoxy)-propylamino]-acetic acid hydrochloride salt;
[2-(4-Methylsufanyl-phenylsufanyl)-ethylamino]-acetic acid tert-butyl ester;
[2-(4-Methylsufanyl-phenylsufanyl)-ethylamino]-acetic acid hydrochloride salt;
(4-Phenyl-butylamino)-acetic acid methyl ester;
4-Phenylbutylamino acetic acid hydrochloride salt;
[2-(3-Chloro-phenoxy)-butylamino]-acetic acid; dihydrochloride;
2-aminomethyl-5-chloro-benzoic acid;
2-aminomethyl-4,5-dichloro-benzoic acid;
2-aminomethyl-3-bromo-benzoic acid;
2-aminomethyl-6-chloro-benzoic acid;
2-(1-aminoethyl)-benzoic acid;
2,3-dihydro-1H-isoindole-4-carboxylic acid;
3-(2-aminomethyl-5-chloro-phenyl)-4H-[1,2,4]oxadiazol-5-one;
(1R,5R,6S)-[6-(aminomethyl)bicyclo[3.2.0]hept-6-yl]acetic acid; and
(1α,3α,5α)-[3-(aminomethyl)bicyclo[3.2.0]hept-3-yl]acetic acid.

11. The method according to claim 6, wherein the opioid receptor antagonist and the alpha2delta ligand are administered substantially simultaneously.

12. The pharmaceutical composition according to claim 1, wherein said alpha2delta ligand is Gabapentin or Pregabalin.

13. The method according to claim 6, wherein the alpha2delta ligand is Gabapentin or Pregabalin.
